(19) United States
(12) Patent Application Publication
(10) Pub. No.: US 2004/0015158 A1
(43) Pub. Date: Jan. 22, 2004

(54) Transilluminator device

(76) Inventors: To-Mu Chen, Chiayi City (TW); Shun-kuai Lee, Tzanghua Shien (TW)

Correspondence Address: RABIN & BERDO, P.C., Suite 500, 1101 14th Street, N.W., Wasington, DC, 20005, US

(21) Appl. No.: 10/197,876

(22) Filed: Jul. 19, 2002

(51) Int. Cl. A61B 18/18

(52) U.S. Cl. 606/11

(57) ABSTRACT

A transilluminator device for locating blood vessels in the tissue. The device includes a plurality of the light emitting diodes with appropriate wavelength. The light emitting diodes emit light through the tissue being examined. This transilluminator device applies the light emitting diodes to emit light into tissue, using the difference of light absorption spectra between tissue and blood vessels to enhence color contrast for locating blood vessels in the tissue. A light intensity adjusting mechanism is intergrated into the transilluminator device to provide the optimal effect individually. The transilluminator device provides the sensors to assure that the light emitting diodes are enabled only when it is in good contact with patient's skin to prevent the physician's eyes from harsh light. The transilluminator device includes a plurality of hook on the basal surface. The hooks comprise a plurality of indentation or a plurality of flange. The operator can utilizes a band to strap the transilluminator device into a section of organic human tissue.

### FIELD OF THE INVENTION

This invention relates to a transilluminator device and, more particularly to a device for transilluminating a section of organic human tissue for locating the blood vessels.

### BACKGROUND OF THE INVENTION

Various medical procedures require transillumination of the skin or other human tissue. For example, premature babies often require intravenous (IV) feeding. Infants have immature vascular development. Obese patients have venous structure that is difficult to penetrate. The difficulties of gaining intravenous access such as for drawing blood, intravenous fusion, etc., are well known to vary from one patient to another. Similarly, for older individuals, certain surgical operations and other medical procedures require the physician to initially determine the location of the patient's veins and other blood vessels. In some patients, the veins are small, deep, and scarcely visible to eyes so that gaining intravenous access is very unpleasant for both the physician and the patient. At the very least, these complications can greatly increase the stress experienced by both the physician and the patient.

In order to determine the most appropriate location to insert the IV device, the children's skin is transilluminated, typically by a conventional fiberoptic cable or halogen lamp. It is important that optical contact must be made through transilluminat ion of this skin between the light source and the physician's sight. Such contact accomplishes at least two things.

1. Optically coupled to skin: If there is no good optical contact with the skin, light will scatter on skin surface and hence reduce transillumination effect. This has negatively impact the efficacy for locating the blood vessels.

2. Harsh to the eye: Where the light source may cause uncomfortable to the eyes if unintentionally fired when not in contact with patient's skin.

Conventional transillumination techniques exhibit several other shortcomings. For example, none of the known devices enables the physician manipulating the distal end of the light sources to adjust the intensity of light simply by a rolling finger on the device.

### SUMMARY OF THE INVENTION

In accordance with the above, this invention, in accordance with one aspect thereof, provides a transilluminator device for use in various medical procedures utilizing light applied through a transparent cover in contact with a section of organic human tissue to locate blood vessels. The apparatus assures good contact between the transparent cover and the section of organic human tissue when the light emitting diodes are triggered. The transilluminator device includes the sensors to pick up the signals once contact with skin at the skin-contacting surface of the transparent cover. These sensors enable the transilluminator device only when the signals detected by the sensors. There are several kinds of the sensors that include tact switche, optical sensor, infrared diodes, phototransistor, photo detector, touch panel, 555 IC and ultrasound sensor or the like. For example, when pressure activates the tact switche and the tact switche triggers the light emitting diodes to fire. In another example, the infrared diodes emit light into the section of organic human tissue then the phototransistors detect the reflex light from skin and trigger the light emitting diodes. The sensors trigger the light emitting diodes by a control circuit. In another example,the sheetmetal be connected to the 555 IC. When a section of organic human tissue contacts with the sheetmetal to trigger the 555 IC. The 555 IC will trigger the light emitting doides to light up.

It is a further object of this invention is to provide a transilluminator device that is safe for use in premature infants and small children. In a preferred embodiment, the transilluminator device includes a plurality of light emitting diodes, which produce less heat and therefore prevent from burning to the patient's skin. The red light emitting diodes (wavelength:600 nm˜650 nm) provide a good color contrast in a section of organic human tissue to locating blood vessels.

It is a further object of this invention is to provide a medical transilluminator that is quick, easy and convenient for virtually all medical personnel to operate. In a preferred embodiment, the transilluminator device includes a plurality of indentation. The operator can utilize a band to strap the transilluminator device to a section of organic human tissue through a plurality of indentation. The transilluminator device comprises a handpiece for a operator holding to locate the blood vessels.

It is a further object of this invention is to provide a transilluminator device, which enables the intensity of the transmitted light to be adjusted in a quick manner and suitable for the physician's eyes by the physician manipulating a rolling wheel on the transilluminator device. In a preferred embodiment, the transilluminator device also includes means for controlling the intensity of light transmitted through a section of organic human tissue. An intensity adjustment wheel which is rotatable is mounted within the handpiece. The wheel maybe comprises a rotary potentiometer, reed potentiometer or potentiometer, which regulate the forward current passing through the light emitting diodes so that a corresponding, predetermined intensity of light is transmitted by the controlling mechanism.

In an alternative embodiment, an intensity adjustment switch which is rotatable is mounted within the handpiece. The operator can adjust the number of the power-on light emitting diodes for controlling the intensity of light transmitted through a section of organic human tissue.

### BRIEF DESCRIPTION OF THE FIGURES

FIG. 1 is a perspective view of a transilluminator device.

FIG. 2 is a cross-sectional view of a transilluminator device.

FIG. 3 is a plan view of a transilluminator device.

FIG. 4 illustrates a patient's hand being positioned over a transilluminator device.

FIG. 5 illustrates a patient holding a transilluminator device.

FIG. 6 illustrates a patient holding a transilluminator device in a cross-sectional view.

FIG. 7 illustrates the tact switches on a transilluminator device.

FIG. 8 is a cross-sectional view of a transilluminator device.

FIG. 9 is a cross-sectional view of an alternative embodiment of the transilluminator device in use.

FIG. 10 is a cross-sectional view of an alternative embodiment of the transilluminator device in use.

FIG. 11 is a schematic block diagram of the electrical circuit for the transilluminator device.

### DETAILED DESCRIPTION

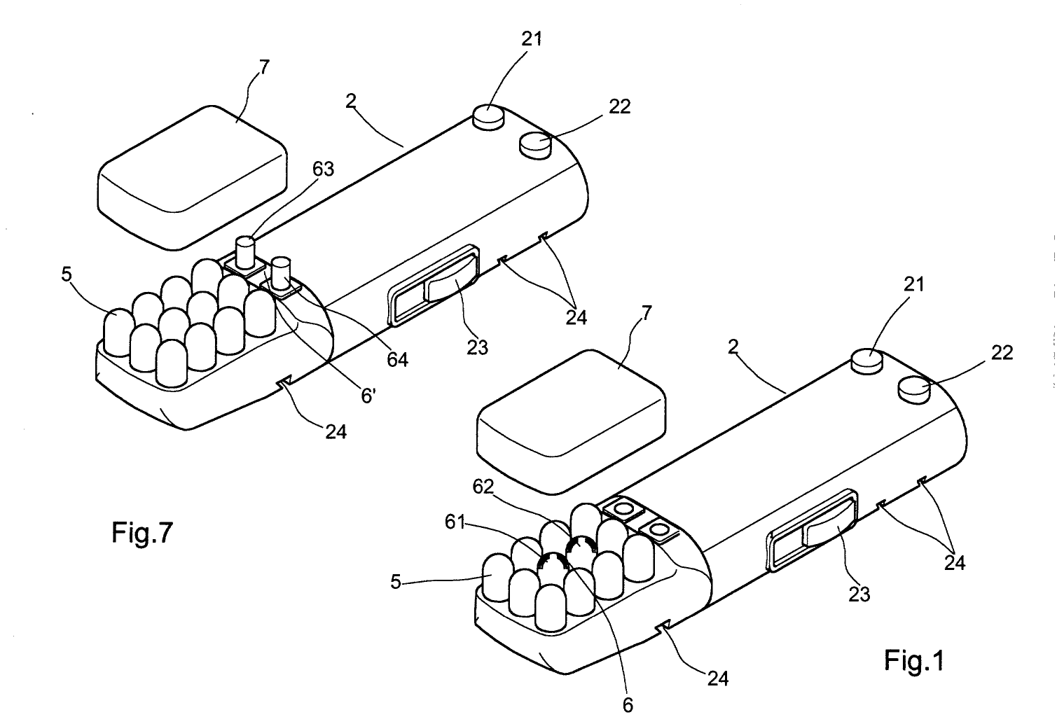

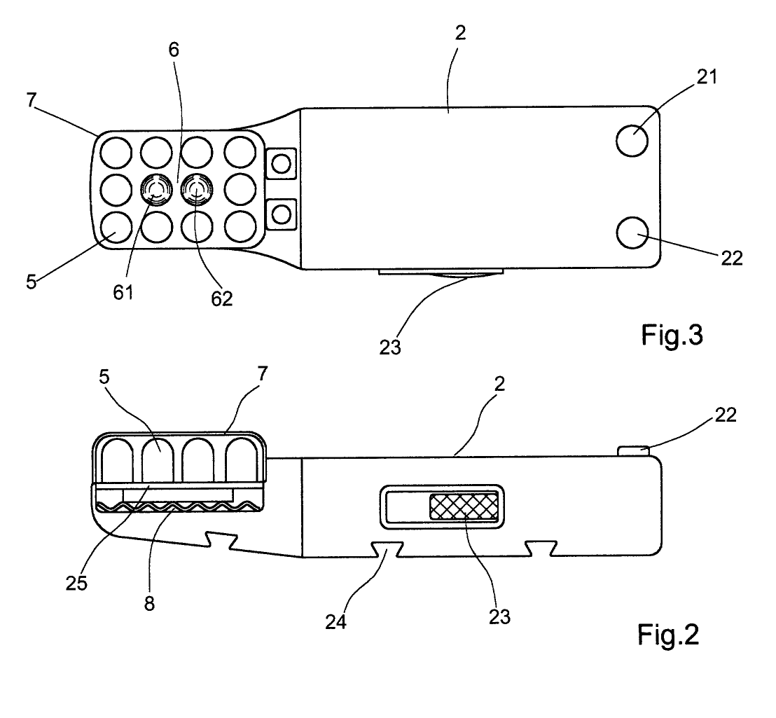

First, please view FIG. 1 FIG. 2 FIG. 3, a power-on switch 21 and a low battery alarm 22, which are located on the handpiece 2. The low battery alarm 22 alarms when the power of the transilluminator device is low. An intensity adjustment wheel 23 which controls the predetermined intensity of light is rotatably mounted on the side of the handpiece 2. The intensity adjustment wheel 23 regulates the forward current passing through the light emitting diodes 5. As a result, a desired intensity of light is transmitted from the transparent cover 7 to the transilluminating a section of organic human tissue 4. A plurality of indentation 24, which are located in the underlayer of the handpiece 2.

A circuitry board 25 is positioned within the handpiece 2. A plurality of the light emitting diodes 5 and the sensors 6 are located on the circuitry board 25, wherein the sensors 6 comprise an infrared diode 61 and a phototransistor 62. A transparent cover 7 caps the light emitting diodes 5 and the sensors 6. The light emitting diodes 5 and the circuitry board 25 are on the heat sink substrate 8. The heat sink substrate 8 eliminates heat generating by the light emitting diodes 5.

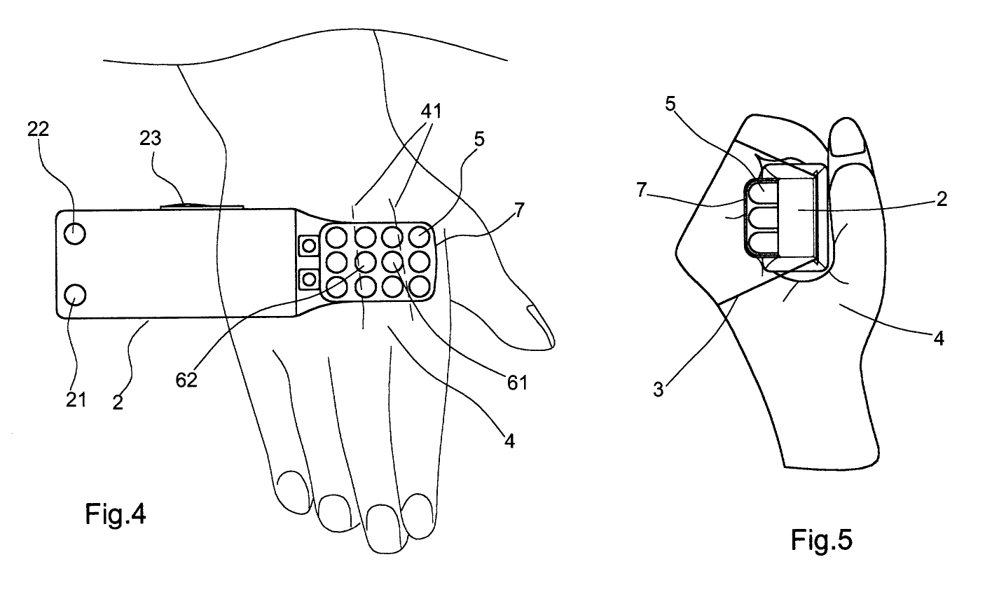

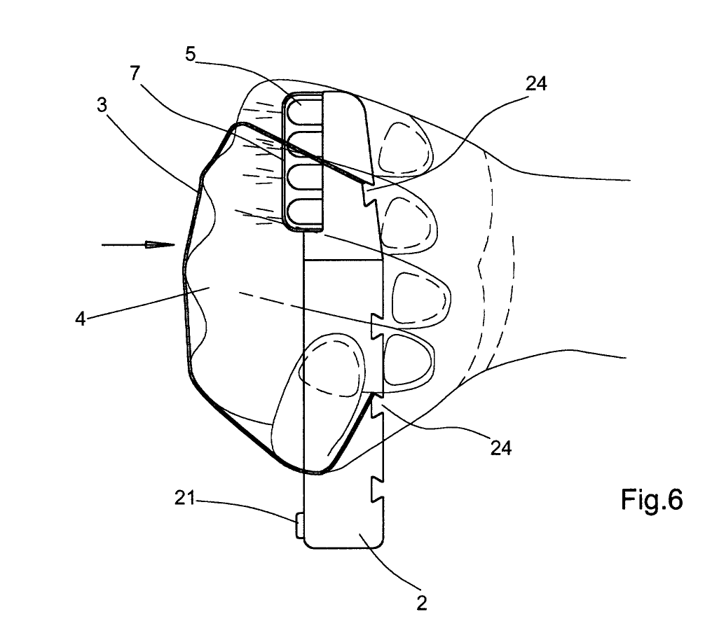

Referring now to FIG. 4, FIG. 5, FIG. 6 when a physician applies the transparent cover 7 to contact a section of organic tissue 4 and straps the handpiece 2 to a section of organic tissue 4 by a plurality of indentation 24 and a band 3. The flanges(not show) also may replace a plurality of indentation 24. A plurality of the light emitting diodes 5 only fires when a section of organic tissue 4 contacts with the transparent cover 7. The sensors 6 which comprise an infrared diode 61 and a phototransistor 62 detect the signals from a section of organics tissue 4. The sensors 6 triggers a plurality of the light emitting diodes 5 to fire when there is good contact with a section of organics tissue 4 and the transparent cover 7. An operator applies a plurality of the light emitting diodes 5 to emit light into a section of organics tissue 4 to produce a difference in light absorption spectra between a section of organics tissue 4 and the blood vessels 41. The difference in light absorption spectra produces color contrast. An operator locates the blood vessels 41 in a section of organic tissue 4 utilizing the color contrast. The light emitting diodes 5 will automatically shut down to prevent the harsh light from illuminating a physician's eyes if unintentionally triggered by the sensors 6 when not in close contact with a section of organic human tissue 4.

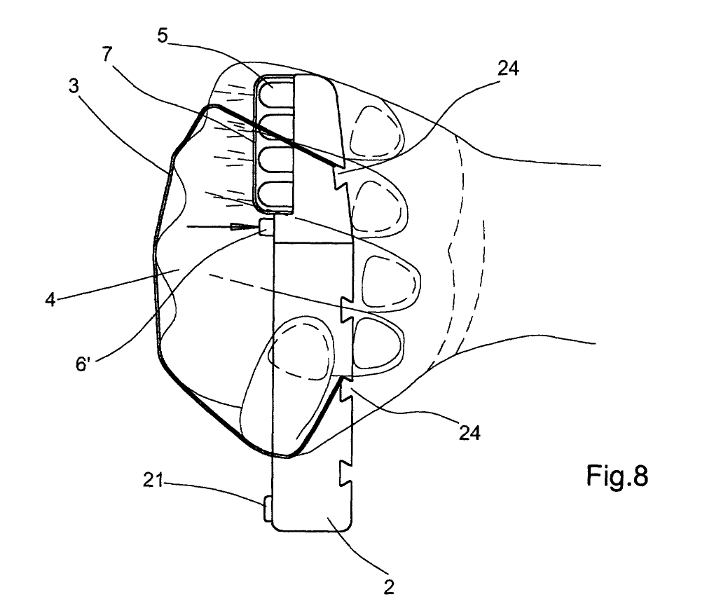

FIG. 7 FIG. 8, illustrating an alternative embodiment of the sensors 6. The sensors 6′ are the tact switches 63-64. When the transparent cover 7 contacts a section of organic human tissue 4 with a certain pressure, the tact switches 63-64 will be switch on and trigger the light emitting diodes 5 to fire.

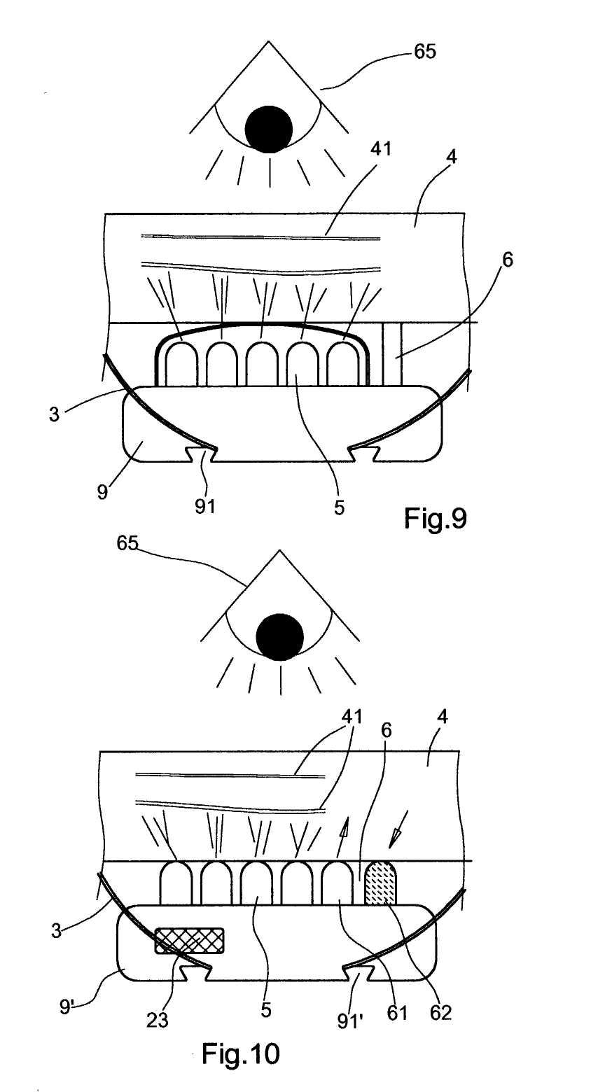

FIG. 9 FIG. 10, illustrating alternative embodiment of the transilluminator. The transilluminator device comprises a plurality of light emitting diodes 5 and the sensors 6. The sensors 6 is located adjacent to the light emitting diodes 5.

A transparent cover 7 caps the light emitting diodes 5. A plurality of indentation 91′ is located in the underlayer of the handpiece 9 for strapping the handpiece 9 to a section of organic human tissue 4 (e.g. a patient's hand) by a band 3. The light emitting diodes 5 fires only when the sensors 6 detect the signals from a section of organic human tissue 4. The sensors 6 may be replaced by the infrared diode 61 and the phototransistor 62. An intensity adjustment wheel 23 is mounted on the side of the handpiece 2, which controls the predetermined intensity of light. The intensity adjustment wheel 23 regulates the forward current passing through the light emitting diodes 5. The operator utilizes the light emitting diodes 5 applied through the transparent cover 7 in contact with a section of organic human tissue 4 for transilluminating a section of organic human tissue 4 (e.g. the skin). The blood vessels 41 are illuminated when a section of organic human tissue 4 is viewed from the position of the eye of an observer 65 to locate the blood vessels 41.

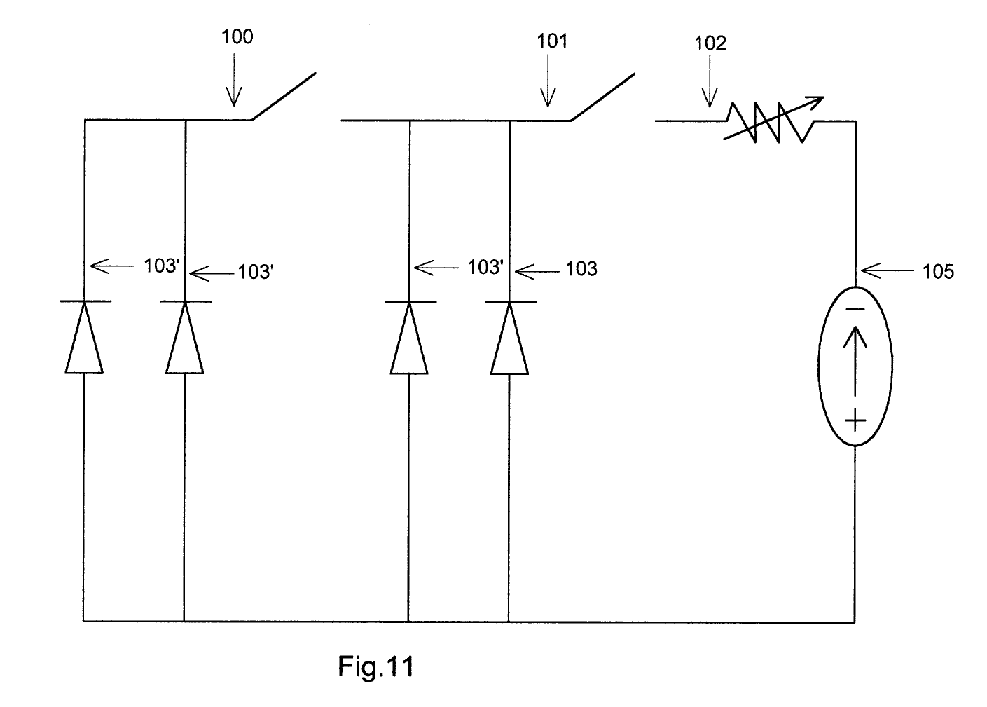

FIG. 11, illustrating the schematic block diagram of the electrical circuit for the transilluminator device. The power source 105 drives the light emitting diode 103, 103′. The tact switch 101 controls the power of the light emitting diode 103, 103′. The operator turns the switch 100 for adjusting the number of the power-on light emitting diodes for controlling the intensity of light transmitted through a section of organic human tissue. The variable resistance 105 can adjust the current passing the light emitting diodes 103, 103′.

From the embodiments of the present invention, it is evident that the objects of the invention are attained. Although the invention has been described and illustrated in detail, it is to be clearly understood that the same is intended by way of illustration and example only and is not to be taken by way of limitation. Accordingly, the spirit and scope of the invention are to be limited only by the terms of the appended claims.

What is claimed is:

1. A transilluminator device for locating blood vessels in a section of organic human tissue, apparatus for assuming contact between the transilluminator device and a section of organic human tissue to prevent the eyes from harsh light, said apparatus comprising:
a plurality of the light emitting diodes and wherein said light emitting diodes emit light beam into a section of organic human tissue for locating blood vessels;
at least a sensor for picking up the signals at a skin-contacting surface of the transilluminator device and enabling application of said light emitting diodes only when the signals from a section of organic human tissue detected by said sensors.

2. The device of claim 1 further including means for adjustment the light intensity of said light emitting diodes.

3. The device of claim 1 further including a plurality of hook. The operator utilizes a band to strap the transilluminator device to a section of organic human tissue by said a plurality of hook.
